(19) United States
(12) Patent Application Publication
(10) Pub. No.: US 2005/0282878 A1
(43) Pub. Date: Dec. 22, 2005

(54) Process for the purification of fluconazole

(76) Inventors: Batchu Chandrasekhar, Navi Mumbai (IN); Ramasubramanian Sridharan, Gujrat (IN); Biju Kumar Gopinathen Pillai, Navi Mumbai (IN); Shekhar Bhaskar Bhirud, Navi Mumbai (IN)

Correspondence Address: DILWORTH & BARRESE, LLP, 333 EARLE OVINGTON BLVD., UNIONDALE, NY, 11553, US

(21) Appl. No.: 11/103,816

(22) Filed: Apr. 12, 2005

Related U.S. Application Data
(60) Provisional application No. 60/561,733, filed on Apr. 13, 2004.

(51) Int. Cl. A61K 31/4196; C07D 43/02

(52) U.S. Cl. 514/383; 548/266.6

(57) ABSTRACT

The present invention relates to processes for the purification of crude fluconazole including at least a first leaching, a second leaching, and an acid/base treatment and to highly purified fluconazole produced from the processes.

### CROSS-REFERENCE TO RELATED APPLICATIONS

This application claims the benefit under 35 U.S.C. §119 to Provisional Application No. 60/561,733, filed Apr. 13, 2004, and entitled “PROCESS FOR THE PURIFICATION OF FLUCONAZOLE”, the contents of which are incorporated by reference herein.

### BACKGROUND OF THE INVENTION

1. Technical Field

The present invention generally relates to processes for the purification of fluconazole.

2. Description of Related Art

The present invention relates to a process for the preparation of fluconazole (also known as 2,4-difluoro-α,α¹-bis-(1H-1,2,4-triazol-1-ylmethyl) benzyl alcohol) of Formula I: 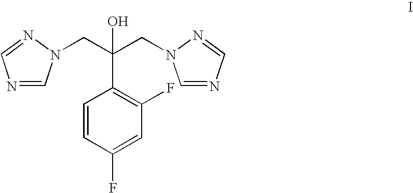
Fluconazole is a synthetic triazole antifungal agent sold under the brand name Diflucan®. Fluconazole is a highly selective inhibitor of fungal cytochrome P-450 sterol C-14 alpha-demethylation. Fluconazole is indicated for the treatment of vaginal candidiasis (vaginal yeast infections due to Candida), oropharyngeal and esophageal candidiasis, and cryptococcal meningitis. See, e.g., The Merck Index, Thirteenth Edition, 2001, p. 728-29, monograph 4148; and Physician's Desk Reference, “Diflucan,” 58^{th} Edition, pp. 2590-2594 (2003).

U.S. Pat. No. 4,404,216 (“the '216 patent”), herein incorporated by reference, discloses fluconazole. The '216 patent further discloses processes for the preparation of fluconazole. These processes for the preparation of fluconazole suffer from numerous drawbacks, most pertinent of which is the production of impurities. Thus, it would be advantageous to reduce the level of impurities in the preparation of fluconazole.

Some impurities that are present in the production of fluconazole include the following which were determined from an HPLC analysis of different batches of fluconazole produced by the reaction of 1-(2,4-difluorophenyl)-2-(1H-1,2,4-triazol-1-yl)ethanone with trimethylsulfoxonium iodide and 1,2,4-triazole in the presence of sodium hydroxide, and identified below in Scheme I: 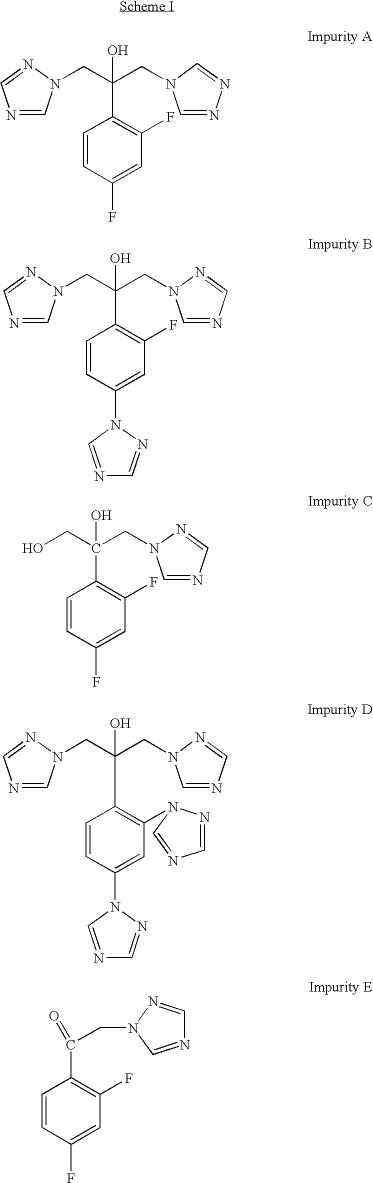
These impurities can be divided into two types: Type 1 impurities (A to D) formed during the synthesis of fluconazole and are characterized as process related and Type 2 impurity (E) related to intermediates of the process used to prepare fluconazole, which is carried into the active pharmaceutical ingredient (API) without conversion. Each of these impurities are typically present in a range of from about 0.1% to about 3.5%.

Impurity A: (R,S)-2-(2,4-difluorophenyl)-1-(1H-1,2,4-triazol-1-yl)-3-(4H-1,2,4-triazol-4-yl) propanol. The structural isomer of fluconazole was present in amounts ranging from about 0.5% to about 1% following synthesis. This impurity forms because the nitrogen at the 4th position of the triazole ring competes with the nitrogen of the 1^{st} position during the reaction with an epoxide. Some amount of equilibrium exists between 1H and 4H forms. This impurity is the most difficult to remove from crude fluconazole.

Impurity B: 2-[2-fluoro-4-(1H-1,2,4-triazol-1-yl)phenyl]-1,3-di 1H-1,2,4-triazol-1-yl) propan-2-ol. This impurity is present in amounts ranging from about 0.1% to about 0.3% following synthesis. A 1,2,4-triazole can react with aromatic fluorine to form this impurity.

Impurity C: (2S)-2-[2,4-difluorophenyl)-3-(1H-1,2,4-triazol-1-yl) propane-1,2-diol. This impurity is present in amounts ranging from about 3% to about 3.5% following synthesis. This dihydroxy impurity forms because hydrolysis of the epoxide occurs during ring opening of the epoxide with 1,2,4-triazole.

Impurity D: 2-(2,4-di-1H-1,2,4-triazol-1-yl phenyl)-1,3-di-1H-1,2,4-triazol-1-yl propan-2-ol. This impurity is present in amounts ranging from about 0.5% to about 1% following synthesis. This impurity forms for one of two reasons, either by disubstitution of aromatic fluorine in fluconazole with 1,2,4-triazole, or impurity B can further react with 1,2,4-triazole to produce disubstituted products.

Impurity E: 1-(2,4-difluoro phenyl)-2-(1H-1,2,4-triazol-1-yl) ethanone. This intermediate is present in amounts ranging from about 0.1% to about 0.3%. This impurity may be present because the intermediate has been unreacted. The unreacted intermediate ends up in the API.

Efforts are made to prepare pharmaceutical products of a high grade and with a minimum amount of impurities present. The control of impurities requires a study of various options to decide upon the reaction conditions and testing protocols necessary to insure that drugs which are administered to the public are substantially pure. Accordingly, there remains a need for an improved process for preparing fluconazole that eliminates or substantially reduces the impurities in a convenient and cost efficient manner to provide highly purified fluconazole.

### SUMMARY OF THE INVENTION

In one embodiment of the present invention, substantially pure fluconazole which comprises less than about 0.05 weight % (R,S)-2-(2,4-difluorophenyl)-1-(1H-1,2,4-triazol-1-yl)-3-(4H-1,2,4-triazol-4-yl) propanol is provided.

In a second embodiment of the present invention, substantially pure fluconazole which comprises less than about 0.05 weight % 2-[2-fluoro-4-(1H-1,2,4-triazol-1-yl)phenyl]-1,3-di 1H-1,2,4-triazol-1-yl) propan-2-ol is provided.

In a third embodiment of the present invention, substantially pure fluconazole which comprises less than about 0.05 weight % (2S)-2-[2,4-difluorophenyl)-3-(1H-1,2,4-triazol-1-yl) propane-1,2-diol is provided.

In a fourth embodiment of the present invention, substantially pure fluconazole which comprises less than about 0.05 weight % 2-(2,4-di-1H-1,2,4-triazol-1-yl phenyl)-1,3-di-1H-1,2,4-triazol-1-yl propan-2-ol is provided.

In a fifth embodiment of the present invention, substantially pure fluconazole which comprises less than about 0.05 weight % 1-(2,4-difluoro phenyl)-2-(1H-1,2,4-triazol-1-yl) ethanone is provided.

In a sixth embodiment of the present invention, substantially pure fluconazole which comprises less than about 0.05 weight % of at least one compound selected from the group consisting of (R,S)-2-(2,4-difluorophenyl)-1-(1H-1,2,4-triazol-1-yl)-3-(4H-1,2,4-triazol-4-yl) propanol, 2-[2-fluoro-4-(1H-1,2,4-triazol-1-yl)phenyl]-1,3-di 1H-1,2,4-triazol-1-yl) propan-2-ol, (2S)-2-[2,4-difluorophenyl)-3-(1H-1,2,4-triazol-1-yl) propane-1,2-diol, 2-(2,4-di-1H-1,2,4-triazol-1-yl phenyl)-1,3-di-1H-1,2,4-triazol-1-yl propan-2-ol, 1-(2,4-difluoro phenyl)-2-(1H-1,2,4-triazol-1-yl) ethanone and mixtures thereof is provided.

In yet another embodiment of the present invention, a pharmaceutical composition is provided comprising substantially pure fluconazole and less than about 0.05 weight % (R,S)-2-(2,4-difluorophenyl)-1-(1H-1,2,4-triazol-1-yl)-3-(4H-1,2,4-triazol-4-yl) propanol.

In yet another embodiment of the present invention, a pharmaceutical composition is provided comprising substantially pure fluconazole and less than about 0.05 weight % 2-[2-fluoro-4-(1H-1,2,4-triazol-1-yl)phenyl]-1,3-di 1H-1,2,4-triazol-1-yl) propan-2-ol.

In yet another embodiment of the present invention, a pharmaceutical composition is provided comprising substantially pure fluconazole and less than about 0.05 weight % (2S)-2-[2,4-difluorophenyl)-3-(1H-1,2,4-triazol-1-yl) propane-1,2-diol.

In yet another embodiment of the present invention, a pharmaceutical composition is provided comprising substantially pure fluconazole and less than about 0.05 weight % 2-(2,4-di-1H-1,2,4-triazol-1-yl phenyl)-1,3-di-1H-1,2,4-triazol-1-yl propan-2-ol.

In yet another embodiment of the present invention, a pharmaceutical composition is provided comprising substantially pure fluconazole and less than about 0.05 weight % 1-(2,4-difluoro phenyl)-2-(1H-1,2,4-triazol-1-yl) ethanone.

In yet another embodiment of the present invention, a pharmaceutical composition is provided comprising substantially pure fluconazole and less than about 0.05 weight % of at least one compound selected from the group consisting of (R,S)-2-(2,4-difluorophenyl)-1-(1H-1,2,4-triazol-1-yl)-3-(4H-1,2,4-triazol-4-yl) propanol, 2-[2-fluoro-4-(1H-1,2,4-triazol-1-yl)phenyl]-1,3-di 1H-1,2,4-triazol-1-yl) propan-2-ol, (2S)-2-[2,4-difluorophenyl)-3-(1H-1,2,4-triazol-1-yl) propane-1,2-diol, 2-(2,4-di-1H-1,2,4-triazol-1-yl phenyl)-1,3-di-1H-1,2,4-triazol-1-yl propan-2-ol, and 1-(2,4-difluoro phenyl)-2-(1H-1,2,4-triazol-1-yl) ethanone.

According to another aspect, the present invention provides a process for the formation of highly purified fluconazole and pharmaceutically acceptable salts thereof from crude fluconazole which comprises (a) a first leaching of crude fluconazole in a first solvent solution comprising water, one or more aliphatic esters and one or more ketones; (b) a second leaching of the product of step (a) with a second solvent solution comprising water, one or more aliphatic esters and one or more ketones; and (c) treating the product of step (b) with an acid/base to obtain highly purified fluconazole.

Yet another aspect of the present invention provides highly purified fluconazole and pharmaceutically acceptable salts thereof having a purity of greater than about 99.5%.

Advantages of the present invention include at least the following:
1. The present invention provides for an improved process which is viable and efficient on a commercial scale and results in highly purified fluconazole and pharmaceutically acceptable salts thereof by a relatively simple process.
2. The present invention provides for improved efficiency because it utilizes inexpensive reagents and produces less waste and fewer impurities.

### DETAILED DESCRIPTION OF THE PREFERRED EMBODIMENTS

One aspect of the present invention is directed to substantially pure fluconazole which comprises less than about 0.05 weight % of at least one compound selected from the group consisting of (R,S)-2-(2,4-difluorophenyl)-1-(1H-1,2,4-triazol-1-yl)-3-(4H-1,2,4-triazol-4yl) propanol, 2-[2-fluoro-4-(1H-1,2,4-triazol-1-yl)phenyl]-1,3-di 1H-1,2,4-triazol-1-yl) propan-2-ol, (2S)-2-[2,4-difluorophenyl)-3-(1H-1,2,4-triazol-1-yl) propane-1,2-diol, 2-(2,4-di-1H-1,2,4-triazol-1-yl phenyl)-1,3-di-1H-1,2,4-triazol-1-yl propan-2-ol, 1-(2,4-difluoro phenyl)-2-(1H-1,2,4-triazol-1-yl) ethanone and mixtures thereof.

Another aspect of the present invention provides a process for the formation of highly purified fluconazole and pharmaceutically acceptable salts thereof from crude fluconazole. It has been found that water mixed with organic solvents advantageously eliminates the impurities found in crude fluconazole, particularly, impurities A to E as discussed above. Accordingly in one embodiment of the present invention, the purification process includes at least the steps of (a) a first leaching of crude fluconazole, (b) a second leaching of the product of the step (a), and (c) an acid/base treatment of the product of step (b).

In step (a), crude fluconazole is added to an aqueous solvent solution including at least water, one or more aliphatic esters and one or more ketones. The aliphatic esters which can be used in the aqueous solvent solution in the process according to the invention can be linear or branched and can contain from 2 to 20 carbon atoms and preferably 2 to 12 carbon atoms. For example, the esters can be of the formula R′COOR wherein R′ and R can be the same or different and can be straight or branched alkyl groups having 1 to 10 carbon atoms. The esters can be derivatives of a carboxylic acid having at least 2 carbon atoms or can be derivatives of an alkanol such as, for example, methanol, ethanol, n-propanol, isopropanol, n-butanol, isobutanol, tert-butanol, and the like. Suitable aliphatic esters include, but are not limited to, ethyl acetate, propyl acetate, isopropyl acetate, butyl acetate, sec-butyl acetate, tert-butyl acetate, ethyl butyrate, ethyl caproate and the like and mixtures thereof.

Suitable ketones for use in the aqueous solvent solution in the process according to the invention include ketones having from 3 to about 12 carbon atoms. Representative examples include, but are not limited to, acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone (methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and the like and mixtures thereof.

In step (a), crude fluconazole is added to a first aqueous solvent solution wherein water may be present in a ratio in the range of from about 1:3 w/v to about 1:6 w/v with respect to the crude fluconazole. The aliphatic ester, e.g., ethyl acetate, may be present in a ratio in the range of from about 1:0.2 w/v to about 1:0.5 w/v with respect to the crude fluconazole. The ketone, e.g., acetone, may be present in a ratio ordinarily ranging from about 1:0.2 w/v to about 1:0.7 w/v with respect to the crude fluconazole. The crude fluconazole and aqueous solvent solution may be heated to a temperature in the range of from about 55° C. to about 65° C. for a time period ordinarily ranging from about 30 to about 60 minutes. When the aqueous solvent solution with respect to crude fluconazole is in the range of from about 1:5:0.2-0.7:0.2-0.7 w/v (water:aliphatic ester:ketone) a suspension is formed. When the ratio of the aqueous solvent solution with respect to crude fluconazole is higher, a solution is formed. The mixture is then cooled to room temperature, a temperature in the range of from about 25° C. to about 30° C. The resulting solid in the suspension is then separated out utilizing conventional techniques, e.g., filtration. Alternatively, when a solution is formed, the purified fluconazole and pharmaceutically acceptable salts thereof can be separated out by crystallization.

In step (b), the second leaching comprises the steps of adding the product obtained from step (a), i.e., the solid, to a second aqueous solvent solution including at least water, one or more aliphatic esters and one or more ketones. The aliphatic ester and ketone employed in the second aqueous solvent solution can be any of the aliphatic esters and ketones described hereinabove and can be the same or different as the aliphatic ester and ketone employed in the first aqueous solvent solution. The water may be present in the second aqueous solvent solution in a ratio of from about 1:3 w/v to about 1:6 w/v with respect to the product of step (a). The aliphatic ester, e.g., ethyl acetate, may be present in the second aqueous solvent solution in a ratio of from about 1:0.2 w/v to about 1:0.5 w/v with respect to the product of step (a). The ketone, e.g., acetone, may be present in the second aqueous solvent solution in a ratio in the range of from about 1:0.2 w/v to about 1:0.7 w/v with respect to the product of step (a). The product of step (a) and the aqueous solvent solution may be heated to a temperature in the range of from about 55° C. to about 65° C. for about 30 to about 60 minutes. When the aqueous solvent solution with respect to the product of step (a) is in the range of from about 1:5:0.2-0.7:0.2-0.7 w/v (water:ethyl acetate:acetone) a suspension is formed. When the ratio is higher, a solution is formed. The mixture is then cooled to room temperature, a temperature in the range of from about 25° C. to about 30° C. The resulting solid in the suspension can then be separated out utilizing conventional techniques, e.g., filtration. The solid obtained can then be washed with chilled water, to further reduce the impurity content. Alternatively, when a solution is formed, the purified fluconazole and pharmaceutically acceptable salts thereof can be separated out by crystallization.

In step (c), an acid/base treatment is performed by, for example (i) dissolving the fluconazole product of step (b) in at least a one or more suitable solvents, e.g. demineralized water, and one or more aqueous acids until the mixture is acidic (e.g. until the pH is about 5.8 to about 8, and preferably about 6.8 to about 7.0) and separating the aqueous layer containing fluconazole, (ii) discarding the organic phase containing impurities, (iii) making the aqueous layer basic by addition of a base, and (iv) dissolving the mixture in at least an organic solvent. In another embodiment, the acid/base treatment can be carried out by, for example, first extracting the aqueous layer containing the fluconazole of step (b) at least three times with an aromatic solvent, e.g., toluene, (about 1:0.5 w/v, fluconazole:toluene) and separating the layers utilizing convention techniques, e.g., filtration, centrifugation, etc. The aqueous layer is decolorized with activated charcoal (about 1:0.1 w/v, fluconazole:activated charcoal). Citric acid (about 1:0.005 w/v, fluconazole:citric acid), acetone (about 1:0.2-0.7 w/v, fluconazole:acetone), ethyl acetate (about 1:0.2-0.7 w/v, fluconazole:ethyl acetate), and methanol (about 1:0.1 w/v, fluconazole:methanol) are then added to the aqueous layer. The aqueous layer is then heated to a temperature in the range of from about 50° C. to about 55° C. The pH of the mixture is adjusted to be within a range of from about 5.8 to about 8, and preferably from about 6.8 to about 7.0. The adjustment of the pH may be performed by slow addition of about 15% base such as ammonia at a temperature in the range of from about 20° C. to about 60° C., and preferably at a temperature of about 50° C. to about 55° C. The mixture should be held at this temperature for a time period of approximately 30 minutes. The mixture is then cooled to a temperature in the range of from about 20° C. to about 30° C., and then further cooled to a temperature in the range of from about 5° C. to about 10° C. The resulting solid can then be filtered. The filtered solid, e.g., a cake, can be washed with water (about 1:0.25 w/v, fluconazole:water). The product can be dried at a temperature of about 65° C. to about 70° C. under vacuum until the moisture content is less than about 0.5%.

By performing the purification processes of the present invention, highly purified fluconazole can be prepared with a degree of purity greater than about 95%, preferably greater than about 97% and most preferably greater than about 99.5%. Also, the impurities in the final product can be reduced by about 70% to about 95% as compared to the crude product. The crude fluconazole for use herein can have a purity as determined by HPLC of less than about 94% and ordinarily can vary in the range of from about 92% to about 94%. After the crude fluconazole is subjected to the first leaching, the known impurities A, B, C, D, and E, as well as unknown impurities were reduced in the range of from about 2.5 times to about 110 times. The first leaching results in the purity of fluconazole of from about 98% to about 99%. After the second leaching, impurity A was reduced from about 0.2% to about 0.1%; impurity B was reduced from about 0.06% to about 0.03%; impurity C was reduced from about 0.3% to about 0.07%. During acid/base treatment, all the major impurities which were greater than about 0.1% were reduced to a level of less than about 0.05%.

According to another aspect, the present invention relates to a pharmaceutical composition comprising the highly purified fluconazole disclosed herein and at least one pharmaceutically acceptable excipient. Such pharmaceutical compositions may be administered to a mammalian patient in any dosage form, e.g., liquid, powder, elixir, injectable solution, etc.

The dosage forms may contain substantially pure fluconazole or, alternatively, may contain substantially pure fluconazole as part of a composition. Whether administered in pure form or in a composition, the substantially pure fluconazole may be in any form. The compositions of the present invention include, but are not limited to, compositions for tableting. Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

Dosage forms may be adapted for administration to the patient by oral, buccal, parenteral, ophthalmic, rectal and transdermal routes. Oral dosage forms include tablets, pills, capsules, troches, sachets, suspensions, powders, lozenges, elixirs and the like. The highly purified form of fluconazole disclosed herein also may be administered as suppositories, ophthalmic ointments and suspensions, and parenteral suspensions, which are administered by other routes. The most preferred route of administration of the fluconazole of the present invention is oral.

Capsule dosages will contain the solid composition within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

The following examples are provided to enable one skilled in the art to practice the invention and are merely illustrative of the invention. The examples should not be read as limiting the scope of the invention as defined in the claims.

### EXAMPLE 1

### Purification of Fluconazole

Step I: First Leaching of Crude Fluconazole:

In a 2 L 4-necked round bottom flask, crude fluconazole (181 g; purity of about 93%), demineralized water (DM water)(905 ml), acetone (81.5 ml), and ethyl acetate (63.5 ml) were added at a temperature in the range of from about 25° C. to about 35° C. under stirring. The reaction mixture was then heated to a temperature in the range of from about 55° C. to about 60° C. and maintained for about 60 minutes. Cold water was circulated to the condenser. Next, the reaction mixture was brought to a temperature in the range of from about 25° C. to about 35° C. in about 60 minutes. This was followed by cooling to a temperature in the range of from about 5° C. to about 10° C. and maintained for about 60 minutes. The precipitated solid was filtered and washed with chilled demineralized water (36 ml). The wet cake weighs about 208 g, with a purity of about 98.96%.

Step II: Second Leaching of Fluconazole:

Into a 2 L 4-necked round bottom flask, the wet fluconazole product obtained from step I (˜208 g; purity of about 98% to about 99%), together with demineralized water (905 ml), acetone (81.5 ml), and ethyl acetate (63.5 ml) were added at a temperature in the range of from about 30° C. to about 35° C. under stirring. The reaction mixture was heated to a temperature in the range of from about 55° C. to about 60° C. and maintained for about 60 minutes. Cold water was circulated to the condenser. Next, the reaction mixture was brought to a temperature in the range of from about 25° C. to about 35° C. in about 60 minutes. This was followed by cooling to a temperature in the range of from about 5° C. to about 10° C. and maintained for about 60 minutes. The precipitated solid was filtered and washed with chilled demineralized water (36 ml). The wet cake weighs about 184 g, with a purity of about 99.45%.

Step III: Acid/Base Treatment of Fluconazole:

Into a 2 L 4-necked round bottom flask, the fluconazole product obtained from step II (˜184 g; purity of about 99.45%), together with demineralized water (543 ml), and concentrated hydrochloric acid (108 ml) were added under stirring at a temperature in the range of from about 25° C. to about 35° C. The reaction mixture was maintained under stirring for about 30 minutes. Toluene (about 100 ml) was added to the reaction mixture and stirred for about 30 minutes. The reaction mixture was then allowed to settle without stirring for about 30 minutes, which allows the layers to separate. The aqueous layer was extracted three times with toluene (100 ml×3). All organic layer washings were clubbed and kept aside for solvent recovery. The aqueous layer was decolorized with activated charcoal (18 g) at a temperature in the range of from about 55° C. to about 60° C. for about 60 minutes. The carbon treated aqueous layer was filtered through a Hyflow bed. The Hyflow bed was washed twice with demineralized water (45 ml×2). Again the washings were clubbed. The aqueous layer was charged into 2 L 4-necked flask. Citric acid (1 g), acetone (81.5 ml), ethyl acetate (81.5 ml), and methanol (18.1 ml) were added, and the mixture was heated to a temperature in the range of from about 50° C. to about 55° C. The pH of the reaction mixture was adjusted to be in the range of from about 6.8 to about 7.0 by slow addition of 15% liquor ammonia (128 ml) over about 60 minutes. The reaction mixture was maintained for about 30 minutes at a temperature in the range of from about 50° C. to about 55° C. The reaction mixture was then cooled to a temperature in the range of from about 25° C. to about 35° C. for about 30 minutes. The reaction mixture was further cooled to a temperature in the range of from about 5° C. to about 10° C. for about 30 minutes. The precipitated product was filtered and washed twice with chilled demineralized water (45 ml×2). The product was dried in a vacuum oven at a temperature in the range of from about 65° C. to about 70° C. until the moisture content was less than about 0.5%. The dried product is the pure fluconazole weighing about 154 g, with a yield of about 85% and purity greater than about 99.5%. All the individual impurities were below about 0.05%.

### EXAMPLE 2

Steps I through III of Example 1 were repeated in substantially the same manner, except that the amounts of solvents were changed as follows:
Step I flask contents: DM water (900 ml), ethyl acetate (36.2 ml), acetone (81.5 ml)
Step II flask contents: DM water (900 ml), ethyl acetate (36.2 ml), acetone (81.5 ml)
Step III flask contents: DM water (543 ml), concentrated HCl (108 ml), acetone (81.5 ml), ethyl acetate (63.3 ml), methanol (18.1 ml)
   The dried product obtained in this example weighed about 145 g, which was a yield of about 80% and had a purity greater than about 99.5%, with all the individual impurities below about 0.05%.

### EXAMPLE 3

Example 2 was repeated in substantially the same manner, except that the amounts of solvents were changed as follows:
Step I flask contents: DM water (900 ml), ethyl acetate (90.5 ml), acetone (81.5 ml)
Step II flask contents: DM water (900 ml), ethyl acetate (90.5 ml), acetone (81.5 ml)
Step III flask contents: DM water (543 ml), concentrated HCl (108 ml), ethyl acetate (63.5 ml), acetone (81.5 ml), methanol (18.1 ml)
   The dried product obtained in this example weighed about 142 g, which was a yield of about 78% and had a purity greater than about 99.5%, with all the individual impurities below about 0.05%.

### EXAMPLE 4

Example 3 was repeated in substantially the same manner, except that the amount of the reactants and solvents were changed as follows:
Step I flask contents: DM water (900 ml), ethyl acetate (63.35 ml), acetone (126 ml)
Step II flask contents: DM water (900 ml), ethyl acetate (63.35 ml), acetone (126 ml)
Step III flask contents: DM water (543 ml), concentrated HCl (108 ml), ethyl acetate (63.5 ml), acetone (81.5 ml), methanol (18.1 ml)
   The dried product obtained in this example weighed about 149 g, which was a yield of about 75% and had a purity greater than 99.5%, with all the individual impurities below about 0.05%.

### EXAMPLE 5

Example 4 was repeated in substantially the same manner, except that the amount of the reactants and solvents were changed as follows:
Step I flask contents: DM water (900 ml), ethyl acetate (63.5 ml), acetone (36.2 ml)
Step II flask contents: DM water (900 ml), ethyl acetate (63.5 ml), acetone (36.2 ml)
Step III flask contents: DM water (900 ml), ethyl acetate (63.5 ml), concentrated HCl (108 ml), acetone (81.5 ml), methanol (18.1 ml)
   The dried product obtained in this example weighed about 148 g, which was a yield of about 81.6% and had a purity greater than 99.5%, with all the individual impurities below about 0.05%.

### EXAMPLE 6

Example 1 was repeated in substantially the same manner, except the pH was adjusted to be in the range of from about 7.8 to about 8.0 with 15% liquor ammonia (135 ml). The dried product obtained in this example weighed about 150 g, which was a yield of about 83% and had a purity greater than 99.5%, with all the individual impurities below about 0.05%.

### EXAMPLE 7

Example 1 was repeated in substantially the same manner except the pH was adjusted to be in the range of from about 5.8 to about 6.0 with 15% aqueous ammonia (120 ml). The dried product obtained in this example weighed about 138 g, which was a yield of about 74% and had a purity greater than 99.5%, with all the individual impurities below about 0.05%.

### EXAMPLE 8

Example 1 was repeated in substantially the same manner except the neutralization temperature was adjusted to a temperature in the range of from about 10° C. to about 15° C. The dried product obtained in this example weighed about 152 g, which was a yield of about 84% and had a purity greater than 99.5%, with all the individual impurities below about 0.05%.

### EXAMPLE 9

Example 1 was repeated in substantially the same manner except the neutralization temperature was adjusted to a temperature in the range of from about 25° C. to about 35° C. The dried product obtained in this example weighed about 143 g, which was a yield of about 79% and had a purity greater than 99.5%, with all the individual impurities below about 0.05%.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A process for preparing purified fluconazole from crude fluconazole comprising the steps of:
(a) a first leaching of crude fluconazole in a first solvent solution comprising water, one or more aliphatic esters and one or more ketones;
(b) a second leaching of the product of step (a) with a second solvent solution comprising water, one or more aliphatic esters and one or more ketones; and
(c) subjecting the product of step (b) to an acid/base treatment to obtain highly purified fluconazole.

2. The process of claim 1, wherein the aliphatic ester of the first and second solvent solution is the same or different and is at least one aliphatic ester having 2 to about 20 carbon atoms.

3. The process of claim 1, wherein the aliphatic ester of the first and second solvent solution is the same or different and is at least one aliphatic ester having 2 to about 12 carbon atoms.

4. The process of claim 1, wherein the aliphatic ester of the first and second solvent solution is the same or different and is selected from the group consisting of ethyl acetate, propyl acetate, isopropyl acetate, butyl acetate, sec-butyl acetate, tert-butyl acetate, ethyl butyrate, ethyl caproate and mixtures thereof.

5. The process of claim 1, wherein the ketone of the first and second solvent solution is the same or different and is at least one ketone having 3 to about 12 carbon atoms.

6. The process of claim 1, wherein the ketone of the first and second solvent solution is the same or different and is selected from the group consisting of acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone (methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and mixtures thereof.

7. The process of claim 1, wherein the aliphatic ester and the ketone of the first and second solvent solution are the same.

8. The process of claim 1, wherein the aliphatic ester and the ketone of the first and second solvent solution are different.

9. The process of claim 1, wherein the aliphatic ester of the first and second solvent solution is ethyl ecetate and the ketone of the first and second solvent solution is acetone.

10. The process of claim 1, wherein the water of the first solvent solution is present in a ratio of about 1:3 w/v to about 1:6 w/v of water to crude fluconazole.

11. The process of claim 1, wherein the aliphatic ester of the first solvent solution is present in a ratio of about 1:0.2 w/v to about 1:0.5 w/v of aliphatic ester to crude fluconazole.

12. The process of claim 1, wherein the ketone of the first solvent solution is present in a ratio of about 1:0.2 w/v to about 1:0.7 w/v of ketone to crude fluconazole.

13. The process of claim 1, wherein the water of the second solvent solution is present in a ratio of about 1:3 w/v to about 1:6 w/v of water to the product of step (a).

14. The process of claim 1, wherein the aliphatic ester of the second solvent solution is present in a ratio of about 1:0.2 w/v to about 1:0.5 w/v of aliphatic ester to the product of step (a).

15. The process of claim 1, wherein the ketone of the second solvent solution is present in a ratio of about 1:0.2 w/v to about 1:0.7 w/v of ketone to the product of step (a).

16. The process of claim 1, wherein the purified fluconazole obtained in a purity of greater than about 97%.

17. The process of claim 1, wherein the purified fluconazole obtained in a purity of greater than about 99.5%.

18. Fluconazole having a purity equal to or greater than about 95%.

19. Fluconazole having a purity equal to or greater than about 97%.

20. Fluconazole having a purity equal to or greater than about 99.5%.

21. A pharmaceutical composition comprising the fluconazole of claim 18 and a pharmaceutically acceptable excipient.

22. A pharmaceutical composition comprising the fluconazole of claim 19 and a pharmaceutically acceptable excipient.

23. A pharmaceutical composition comprising the fluconazole of claim 20 and a pharmaceutically acceptable excipient.

24. Substantially pure fluconazole which comprises less than about 0.05 weight % of at least one compound selected from the group consisting of (R,S)-2-(2,4-difluorophenyl)-1-(1H-1,2,4-triazol-1-yl)-3-(4H-1,2,4-triazol-4-yl) propanol, 2-[2-fluoro-4-(1H-1,2,4-triazol-1-yl)phenyl]-1,3-di 1H-1,2,4-triazol-1-yl) propan-2-ol, (2S)-2-[2,4-difluorophenyl)-3-(1H-1,2,4-triazol-1-yl) propane-1,2-diol, 2-(2,4-di-1H-1,2,4-triazol-1-yl phenyl)-1,3-di-1H-1,2,4-triazol-1-yl propan-2-ol, and 1-(2,4-difluoro phenyl)-2-(1H-1,2,4-triazol-1-yl) ethanone.

25. A pharmaceutical composition comprising the substantially pure fluconazole of claim 24.
